(19) United States
(12) Patent Application Publication
(10) Pub. No.: US 2016/0220477 A1
(43) Pub. Date: Aug. 4, 2016

(54) COSMETIC OR DERMATOLOGICAL USE OF AN EXTRACT OF TAPIRIRA GUIANENSIS

(71) Applicants: BASF Beauty Care Solutions France SAS, Lyon (FR); Centre National de la Recherche Scientifique, Paris (FR); Universite Claude Bernard Lyon I, Villeurbanne (FR)

(72) Inventors: Valérie ANDRE-FREI, Ampuis (FR); Nicolas BECHETOILLE, Brignais (FR); Morgan DOS SANTOS, Saint Verand (FR); Patricia ROUSSELLE, Lyon (FR)

(21) Appl. No.: 15/021,292

(22) PCT Filed: Sep. 11, 2014
(86) PCT No.: PCT/FR2014/052254

§ 371 Date: Mar. 11, 2016

(30) Foreign Application Priority Data
Sep. 12, 2013 (FR) 1358756

(51) Int. Cl. A61K 8/97; C12N 5/071; A61Q 19/08

(52) U.S. Cl. CPC A61K 8/97; A61Q 19/08; C12N 5/0629; C12N 2500/76; C12N 2501/00

(57) ABSTRACT

The present invention relates to the cosmetic use by the topical route of a Tapirira guianensis extract, for stimulating the expression of perlecan and/or dystroglycan and/or VE-cadherin and/or claudin-5, in particular in the extracellular matrix and/or in the epithelial basement membrane, especially the dermoepidermal junction.

It also relates to a cosmetic care method comprising the application, to at least one concerned area of healthy skin and/or healthy mucous membrane and/or healthy scalp, of a Tapirire guianensis extract or of a cosmetic composition comprising such an extract for stimulating the expression of perlecan and/or dystroglycan and/or VE-cadherin and/or claudin-5, in particular in the extracellular matrix and/or in the epithelial basement membrane, especially the dermoepidermal junction.

In addition, it relates to a cosmetic composition comprising a Tapirira guianensis extract and a topically acceptable cosmetic excipient.

Finally, it relates to a Tapirira guianensis extract or a dermatological composition comprising a Tapirira guianensis extract and a dermatologically acceptable excipient for use by the topical route in the treatment and/or prevention of rosacea, telangiectasia and/or pathologies of the oral and/or ocular mucous membranes.

The invention relates to the cosmetic or dermatological use by the topical route of an extract of Tapirira guianensis for stimulating the expression of perlecan and/or dystroglycan and/or collagen XVIII and/or VE-cadherin and/or claudin-5, in particular in the extracellular matrix and/or in the epithelial basement membrane, especially the dermoepidermal junction, and/or to the cosmetic composition which comprises it.

Among the heparan sulfate proteoglycans (HSPGs) of the basal laminae, perlecan has a major role in the morphogenesis of the epithelium, in particular the epidermis, but also in the survival, proliferation and differentiation of keratinocytes and of endothelial cells, in particular skin keratinocytes and endothelial cells. Perlecan regulates these processes by controlling the bioavailability of growth factors.

Dystroglycan is a glycoprotein which is a potential receptor for perlecan. Perlecan and dystroglycan are expressed on basement membranes, which are ubiquitous structures located in various tissues, such as epithelia and endothelia, but also in various cell types, such as keratinocytes, fibroblasts and endothelial cells. They interact together and contribute to ensuring the solidity and stability of the structure of the skin, of the mucus membranes and of the scalp, in particular the epithelium, and especially the epidermis and the dermis. In point of fact, during aging, in particular chronobiological aging, the expression of perlecan and dystroglycan is drastically reduced.

In particular, the expression of perlecan decreases strongly in the dermoepidermal junction and the dermal capillaries with age. This decrease is not due to degradation of the protein but to a decrease in its expression, and very particularly in its transcriptional regulation, by keratinocytes and endothelial cells. Moreover, the inventors have found that a correlation exists between the lack of synthesis of perlecan and the thickness of the skin and/or of the mucous membranes and/or of the scalp, in particular of the epithelium, preferentially the epidermis. The expression of perlecan and dystroglycan is thus of great importance in skin and/or mucous membrane and/or scalp homeostasis and in maintaining their firmness and their density, which are degraded during aging, in particular chronobiological aging.

Collagen is a constituent protein of the extracellular matrix (ECM) present in a large amount in vertebrate tissues. It involves a broad family comprising 29 different types, including collagen XVIII. The latter is known to bind to heparan sulfate proteoglycans (HSPGs) and might consequently participate in the morphogenesis of the epithelium, in particular of the epidermis, more particularly in the mucous membranes.

The cadherins are a family of glycoproteins, including VE-cadherin, expressed at the surface of the endothelial cells in particular. VE-cadherin plays an important role in cell adhesion in tissues and thus in intercellular junctions.

Likewise, the claudins are constituent transmembrane proteins of “tight” junctions, which play a role in cell adhesion.

These two families of proteins are necessary for the survival of cells and have an effect on the retention of fluids and thus on cellulite and periorbital bags.

Surprisingly, the inventors have discovered that a Tapirira guianensis extract stimulates the expression of HSPGs and glycoproteins of basal laminae, and more particularly of perlecan and of dystroglycan, in particular in the extracellular matrix and/or in the epithelial basement membrane, especially the dermoepidermal junction, more particularly in keratinocytes and/or endothelial cells of the skin and/or mucous membranes and/or scalp, preferentially skin keratonicytes and/or endothelial cells. Targeting both keratinocytes and endothelial cells, which are in particular cutaneous, makes it possible to have a two-fold effect: 1) on improving the microvascular pathway for nourishing the skin, the mucous membranes and/or the scalp, and 2) on improving the complexion in terms in particular of radiance.

Targeting not only perlecan but also dystroglycan makes it possible, in addition, to act completely on the pathway of expression and activity of perlecan.

The inventors have also discovered that the Tapirira guianensis extract increases the expression of VE-cadherin and claudin-5, in particular in the dermoepidermal junction, more particularly in keratinocytes and/or endothelial cells, more particularly still in endothelial cells, of the skin and/or mucous membranes and/or scalp, preferably skin keratinocytes and/or endothelial cells. This extract thus makes it possible to decrease, suppress or prevent the retention of fluids and thus the appearance of cellulite and periorbital bags.

Finally, the inventors have discovered that the Tapirira guianensis extract increases the expression of collagen XVIII.

Tapirira guianensis, which belongs to the Sapindales order and to the Anacardiaceae family, is a tree which grows widely in South America, in particular in Amazonia and in particular in French Guyana.

Some parts of the tree have been described in the context of specific treatments in the local pharmacopeia. Thus, it is commonly used as anti-infective administered orally. Likewise, the use of its bark on oral and vaginal mucous membranes in specific pharmaceutical applications has already been described, in particular as a cold macerated product or as a decoction administered as a hemostatic mouthwash for combating diarrhea and vomiting or administered by application to the vaginal mucous membranes in order to prevent vaginal hemorrhages and infections resulting from childbirth. Finely grated and pressed, the bark is also used in gargling against benign oral thrush (Etude phytochimique de plantes amazoniennes d'activité antiplasmodiale [Phytochemical study on Amazonian plants having antiplasmodial activity], Vincent ROUMY, July 2007).

However, none of the properties described in the prior art made it possible to predict its advantages in a cosmetic composition or its properties of stimulating the expression of perlecan and/or dystroglycan and/or VE-cadherin and/or claudin-5 and/or collagen XVIII.

The present invention thus relates to the cosmetic or dermatological use by the topical route of a Tapirira guianensis extract for stimulating the expression of perlecan and/or dystroglycan and/or VE-cadherin and/or claudin-5 and/or collagen XVIII, in particular in the extracellular matrix and/or in the epithelial basement membrane, especially the dermoepidermal junction.

Preferably, the use according to the invention is cosmetic and by topical application to at least one concerned area of healthy skin and/or healthy mucous membrane and/or healthy scalp, in particular of a human being.

Preferably, the Tapirira guianensis extract is obtained from the aerial parts, in particular the leaves, and is preferably obtained by aqueous extraction.

Preferably, the use of Tapirira guianensis stimulates the expression of perlecan and/or dystroglycan in keratinocytes and/or endothelial cells of the skin and/or mucous membranes and/or scalp, preferably endothelial cells of the skin.

The present invention also relates to a cosmetic composition comprising a Tapirira guianensis extract and a topically acceptable cosmetic excipient.

Within the meaning of the present invention, the term “cosmetic use and/or composition” is understood to mean a nonpharmaceutical use and/or composition, that is to say a use and/or composition which is not intended for a therapeutic employment and which is targeted at improving the attractiveness and/or the comfort and which is carried out on/applied to a healthy part of the body, in particular healthy mucous membrane, healthy skin and/or healthy scalp.

Within the meaning of the present invention, the term “healthy skin”, “healthy mucous membrane” or “healthy scalp” is understood to mean an area of skin, of mucous membrane or of scalp to which the extract according to the invention is applied and which is said to be “nonpathological” by a dermatologist, that is to say not exhibiting skin infection, disease or condition, such as thrush, impetigo, psoriasis, eczema, acne or dermatitis, or wounds or injuries.

Within the meaning of the present invention, the term “topically acceptable cosmetic and/or dermatological” is understood to mean an ingredient which is appropriate for application by the topical route, which is nontoxic, which does not irritate the skin and/or mucous membranes, which does not induce an allergic response and which is not unstable chemically.

Within the meaning of the present invention, the term “application by the topical route” of an ingredient is understood to mean the direct local application of the ingredient to and/or the spraying of the ingredient over the surface of the skin and/or mucous membranes and/or scalp.

According to the invention, the term “mucous membrane(s)” denotes the ocular mucous membrane, the vaginal mucous membrane, the urogenital mucous membrane, the anal mucous membrane, the nasal mucous membrane and/or the oral mucous membrane, in particular oral, labial and/or the gingival mucous membrane, preferably the ocular and/or oral mucous membranes and preferably again the labial and/or ocular mucous membranes.

The Tapirira guianensis plant extract according to the invention can be extracted from the entire plant or from one or more parts of the plant, chosen in particular from the root, stem, bark, flower, seed, germ, fruit and/or leaf and their mixtures. The extract according to the invention is preferably extracted from the upper parts of the plant, namely the fruit, leaves and branches, and more preferably from the aerial parts, that is to say the leaves and branches. Particularly advantageously, the active ingredient according to the invention is an extract of Tapirira guianensis leaves. This is because the leaves constitute the part of the plant which exhibits the maximum activity and their harvesting comes within a sustainable development approach.

The extract according to the invention can then be obtained by plant extraction methods known in the field, for example by maceration of at least one part of the plant, preferably between 1 and 10% (w/w) in a solvent or a mixture of solvents, preferably a polar protic solvent, and advantageously in water, an alcohol, a glycol, a polyol or a water/alcohol, water/glycol or water/polyol mixture (such as water as a mixture with ethanol, glycerol, butylene glycol or other glycols, such as xylitol, and the like) of 100/0 to 0/100 (v/v). The extract is preferably obtained by aqueous extraction.

Within the meaning of the present invention, the term “Tapirira guianensis extract obtained by aqueous extraction” is understood to mean any extract obtained by extraction with an aqueous solution containing more than 60% by weight, advantageously at least 70% by weight, in particular at least 80% by weight, more particularly at least 90% by weight or particularly at least 95% by weight of water, with respect to the total weight of the aqueous solution, more advantageously still not containing butylene glycol, in particular not containing alcohol and more particularly containing only water.

According to an advantageous embodiment, the extract according to the invention is obtained by extraction at a temperature of between 0° C. and 30° C., in particular by cold maceration, preferably at 4° C., or at ambient temperature, that is to say between 18 and 25° C., preferably 20° C., optionally after a state of drying the plant.

According to a preferred embodiment, the extraction according to the invention is obtained by maceration for a period of time of between 30 minutes and 24 hours, preferably between 1 and 20 hours, more preferably between 2 and 16 hours, in particular 16 hours.

According to an advantageous embodiment, the extract according to the invention is an extract obtained by cold maceration and/or by maceration at ambient temperature, preferably of a mixture of stem and leaves, more preferably still an extract of leaves, optionally after a stage of drying the plant.

According to a particularly advantageous embodiment, the Tapirira guianensis extract according to the invention is obtained from the aerial parts and advantageously the leaves, with cold extraction in an aqueous, butylene glycol or aqueous/alcoholic medium, preferably in water, preferably carried out under the following conditions:
at a temperature preferably between 0 and 20° C., in particular between 0 and 10° C. and advantageously at approximately 4° C.

and/or
for a period of time of between 2 and 20 hours, advantageously between 4 and 20 hours, preferably approximately 16 hours.

This is because this extraction process exhibits the advantage of providing an active ingredient with a maximum activity in stimulating the expression of HSPGs, in particular of perlecan, dystroglycan and/or collagen XVIII and/or with an activity in increasing the expression of VE-cadherin and/or claudin-5.

The extract is then dissolved in an aqueous vehicle, preferably water. The extract is subsequently used in accordance with the present invention, optionally after filtration.

The extract obtained is subsequently preferably centrifuged and/or filtered and/or distilled in order to recover the active soluble fraction. It is preferably filtered at a cutoff threshold of 0.45 μm, more preferably 0.22 μm. Additional stages of decoloration and/or deodorization can be carried out on the extract at any stage of the extraction and according to techniques known to a person skilled in the art.

The extract according to the invention can also be subsequently concentrated by evaporation of the solvent, for example by freeze drying or by atomization.

Particularly advantageously, the amount of plant during the extraction, preferably the upper parts of the plant, advantageously the aerial parts, in particular the leaf, is 1% by weight with respect to the total weight of the plant/extraction solvent mixture, the solvent preferably being an aqueous solution. In particular, the plant is ground before the extraction.

The extract obtained is preferably soluble and/or dissolved in a solvent, in particular a polar solvent, such as water, an alcohol, a polyol, a glycol or one of their mixtures, preferably an aqueous/glycol mixture, more preferably containing a glycol chosen from caprylyl glycol, pentylene glycol, hexylene glycol and their mixtures.

Advantageously, the extract is dissolved and/or soluble in an aqueous solution containing hexylene glycol and/or pentylene glycol, in particular containing between 0.1 and 10% by weight of hexylene glycol and/or of pentylene glycol with respect to the total weight of the aqueous solution, more particularly between 1 and 5% by weight of hexylene glycol and/or of pentylene glycol with respect to the total weight of the aqueous solution. Advantageously, the extract is soluble in an aqueous solution containing caprylyl glycol, in particular containing between 0.01 and 5% by weight of caprylyl glycol with respect to the total weight of the aqueous solution, more particularly between 0.1 and 1% by weight of caprylyl glycol with respect to the total weight of the aqueous solution. Advantageously, the aqueous solution does not contain butylene glycol. Particularly advantageously, the extract thus obtained is adjusted and/or maintained at a neutral pH in order to avoid increasing the HI index, which appears undesirable with an extract having a basic pH.

Within the meaning of the present invention, the term “stimulation of the expression of perlecan and/or dystroglycan and/or VE-cadherin and/or claudin-5 and/or collagen XVIII” is understood to mean an increase in the gene and/or protein expression respectively of perlecan and/or dystroglycan and/or VE-cadherin and/or claudin-5 and/or collagen XVIII, preferably in the gene and/or protein expression of perlecan and/or dystroglycan and/or VE-cadherin and/or claudin-5, more preferably in the protein expression. This increase can be measured on a model comprising at least one cell type exhibiting an expression of perlecan and/or dystroglycan and/or VE-cadherin and/or claudin-5 and/or collagen XVIII, advantageously keratinocytes and/or endothelial cells, preferably skin keratinocytes and/or endothelial cells, on contact with the Tapirira guianensis extract according to the invention and is reflected by an increase in the respective gene and/or protein expression of perlecan and/or dystroglycan equal to or greater than 10%, advantageously equal to or greater than 20%, with respect to the level of gene and/or protein expression in a control model, that is to say without being brought into contact with the Tapirira guianensis extract according to the invention. The increase in this expression is preferably protein expression. Such models are described in the examples.

Advantageously, the use according to the present invention is for preventing and/or combating aging of the skin and/or mucous membranes and/or scalp, in particular chronobiological and/or photobiological aging, for preventing and/or combating the decrease in homeostasis of the skin and/or mucous membranes and/or scalp and/or for improving it, in particular in the epidermis, for reinforcing the epithelial basement membrane of the skin and/or mucous membranes and/or scalp, preferably the dermoepidermal junction, for improving keratinocyte proliferation and/or differentiation, especially at the epidermal level, in particular associated with aging of the skin and/or mucous membranes and/or scalp, for preventing and/or combating a decrease in vascularization of the skin and/or mucous membranes and/or scalp and/or for improving it, in particular for improving the structure of the capillaries of the skin and/or mucous membranes and/or scalp, in particular skin capillaries, for improving the morphogenesis of the epithelium of the skin and/or mucous membranes and/or scalp, preferably the epidermis, for restoring the epithelial architecture of the skin and/or mucous membranes and/or scalp, preferably the epidermal architecture, in particular of skin and/or mucous membranes and/or scalp having undergone aging, in particular chronobiological aging, for improving the complexion of the skin and/or mucous membranes, especially making it uniform, for improving the firmness and/or the density of the skin and/or mucous membranes and/or scalp, and/or for combating the decrease in the thickness of the epithelium of the skin and/or mucous membranes and/or scalp, preferably the epidermis, and/or for increasing the thickness of the epithelium of the skin and/or mucous membranes and/or scalp, preferably the epidermis, and/or for the treatment or prevention of cracks and/or for combating, treating and/or preventing water retention and/or cellulite and/or periorbital bags and/or for increasing and/or maintaining cell adhesion.

In a particular embodiment, the purpose of the use according to the present invention is to improve the complexion of the skin, advantageously by eliminating red patches and/or by making the complexion uniform and/or by giving it a luminous, radiant, healthy and/or nourished appearance, a well-looking effect and/or a pinkish radiance.

This is because the radiance of the complexion generally reflects a healthy condition of the skin. Numerous intrinsic or extrinsic factors can cause a non-uniform, muddy complexion. Among the factors which affect the radiance of the complexion of the skin, mention may be made of stress, fatigue, hormonal changes, dehydration of the epithelium, preferably the epidermis, polluting agents and chronobiological and photobiological aging. These factors tend to make the complexion muddy, and make it non-uniform, dull, waxy, indeed even sickly. The expression of perlecan and dystroglycan has an impact on the microvascular pathway, thereby making it possible to nourish the skin better and to detoxify it better and thus to give it a nourished and healthy appearance. In addition, the color of the skin is influenced by the microcirculation: on reaching the microvessels, light comes into contact with the red blood cells which specifically absorb in the green region. Red is reflected at the surface, giving the skin a pinkish complexion and therefore a pinkish radiance. The radiance of the complexion also depends on the reflecting capacity of the skin. This reflecting capacity is influenced by the texture of the skin. Skin which has a softer, more flexible texture will thus have a better respect. The restoration of the epidermal architecture and the improvement in the morphogenesis of the epithelium, preferably the epidermis, obtained by virtue of the stimulation of the expression of perlecan and/or dystroglycan will thus also make it possible to improve the skin complexion.

The improvement in the radiance or glow of the complexion can in particular be measured by an objective instrumental method. This in vivo method of measurement consists in taking high-resolution photographs, with cross-polarized light, of the face of volunteers taken at 45° before and after application of the tested product. On the basis of these digital photographs, an image analysis makes it possible to extract and to quantify specific parameters (for example: L*, a*, b*, C, h°) related to the color, radiance, uniformity and texture of the skin.

Likewise, the gloss can in particular be measured according to this method on the basis of high-resolution photographs, with cross-polarized and parallel-polarized light, of the face of volunteers taken at 45° before and after application of the tested product. On the basis of these digital photographs, an image analysis makes it possible to extract and to quantify specific parameters related to the gloss, such as the specular gloss and the contrast gloss.

In another specific embodiment, the purpose of the use according to the present invention is to prevent and/or combat aging of the skin and/or mucous membranes and/or scalp, in particular chronobiological or photobiological aging, by decreasing or suppressing wrinkles and/or fine lines, in particular for mature skin and/or skin exhibiting the first signs of aging.

Within the meaning of the present invention, the term “mature skin” is understood to mean the skin of women or men who are at least 50 years old, advantageously the skin of menopausal women.

Within the meaning of the present invention, the term “skin showing the first signs of aging” is understood to mean the skin of women or men who are between 28 and 40 years old, advantageously skin exhibiting the first expression wrinkles.

In yet another specific embodiment, the purpose of the use according to the present invention is to prevent and/or combat the decrease in the homeostasis of the skin and/or mucous membranes and/or scalp and/or to improve it, in particular in the epidermis.

Homeostasis, especially cutaneous homeostasis, and in particular epidermal homeostasis, results from a finely regulated balance between the processes of proliferation and differentiation of the cells of the skin and in particular of the keratinocytes. These proliferation and differentiation processes participate in the renewal and/or in the regeneration of the skin and result in the maintenance of an unvarying thickness of the skin and in particular of an unvarying thickness of the epithelium, preferably the epidermis. This homeostasis also participates in the maintenance of the mechanical properties of the skin, mucous membranes and scalp.

However, this cutaneous homeostasis can be impaired by certain physiological factors (age, menopause, hormones, stress, and the like) and extrinsic factors (polluting agents, and the like). The regenerative potential of the epithelium, in particular the epidermis, becomes lower: the cells of the basal layer divide less actively, resulting in particular in a slowing down and/or a reduction of epidermal renewal. Consequently, cell renewal no longer compensates for the loss of cells eliminated at the surface, resulting in atrophy of the epithelium, in particular the epidermis, and/or a decrease in the thickness of the skin and/or mucous membranes and/or scalp and/or a loss of density and/or of firmness of the skin and/or mucous membranes and/or scalp. This phenomenon can be accentuated by the menopause. The hormonal deficiencies associated with the menopause are accompanied in particular by a fall in metabolic activity, which might result in a decrease in keratinocyte proliferation and an increase in epidermal differentiation. The use according to the present invention therefore makes it possible to promote homeostasis in order to maintain and/or increase the thickness of the skin and/or mucous membranes and/or scalp and thus to maintain and/or improve the mechanical properties of the skin and/or mucous membranes and/or scalp and/or to improve the firmness and/or the density of the skin and/or mucous membranes and/or scalp, in particular in menopausal women.

The increase in the thickness of the epithelium, preferably the epidermis, can in particular be evaluated ex vivo on a skin and/or mucous membrane and/or scalp biopsy model under survival conditions. The epithelium, preferably the epidermis, is measured at the beginning of the experiment before the application of the test product and at the end of the test period in the presence of the test product, for example 4 days, preferably 7 days. The thickness of the epithelium, preferably the epidermis, is said to be increased if the measurement after application of the product is equal to or greater than 5% at the end of the test period, preferably equal to or greater than 10%.

In yet another specific embodiment, the purpose of the use according to the present invention is to combat, treat and/or prevent water retention and/or cellulite and/or periorbital bags and/or to increase and/or to maintain cell adhesion, in particular for the purpose of combating, treating and/or preventing periorbital bags.

This is because periorbital bags have always been regarded as unsightly and it has always been desired to conceal them, indeed even to remove them. In point of fact, the inventors have discovered that the extract according to the invention has an effect on the decrease in vasodilatation and in excessive edema due to lymphatic stasis, in particular in the periorbital area, and thus on the prevention or treatment of periocular bags.

The term “periorbital bag” is understood to mean, within the meaning of the present invention, an area localized around the eye, in particular below and on the inner side of the eye, exhibiting a relief which is not in the continuity of the skin of the face, that is to say as a hollow or as a bag (swollen area).

A periorbital bag can be differentiated from a dark circle in that it has a different volume. Dark circles are also generally darker than bags. Their location and appearance are different. Their stability and their development over time are different.

They are identified by clinical examination in three phases: a phase of observation, a phase of palpation and a phase of questions.

The effect on the periorbital bags can be measured by simple visual observation or by comparative image analysis.

Advantageously, the concerned area of the skin, in particular of a human being, to which the Tapirira guianensis extract according to the invention or a cosmetic or dermatological composition comprising it is applied is chosen from the face, neck, neckline, bust and/or hands, and very particularly the nasolabial folds, and/or the periorbital area, in particular on the dark circles and crow's feet and/or the outline of the lips and/or the forehead. However, the extract can also be applied to the body and in particular to the stomach, thighs, hips, buttocks and/or waist, areas of the body which can show a loss of firmness and/or density. In addition, in the case of the treatment for combating and/or preventing periorbital bags, the area of application is obviously the periorbital area.

According to the invention, the Tapirira guianensis plant extract according to the invention is used alone or in a cosmetic or dermatological composition at a concentration of between 1×10⁻⁴ and 10% by weight, advantageously between 1×10⁻⁴ and 5% by weight and more particularly between 1×10⁻³ and 3% by weight, with respect to the weight of the total composition, in particular between 0.001 and 0.1% by weight, with respect to the total weight of the composition. It can also be present in a content of between 0.01 and 10% by weight, with respect to the total weight of the composition.

In one embodiment according to the present invention, the Tapirira guianensis extract is used for the manufacture of a dermatological composition for the dermatological care and/or treatment of rosacea or telangiectasia and/or for the care and/or treatment of pathologies of the oral and/or ocular mucous membranes, in particular for the care and/or treatment of pathologies of the oral mucous membranes involving a loss of firmness and/or of density in the oral mucous membrane and/or for improving the oral vascular structure and/or for the care and/or treatment of pathologies of the ocular mucous membranes involving a loss of firmness and/or of density in the ocular mucous membrane and/or for improving the ocular vascular structure and/or for the treatment of pathologies involving an excessive or pathological deterioration in cell adhesion and/or of pathologies related to the retention of water or fluids.

In another embodiment according to the present invention, the Tapirira guianensis extract according to the invention is present in a cosmetic or dermatological composition comprising a topically acceptable cosmetic or dermatological excipient.

This cosmetic or dermatological composition is advantageously intended for application by the topical route.

The cosmetic or dermatological compositions according to the invention thus contain a topically acceptable cosmetic or dermatological excipient in addition to the extract according to the invention. This excipient is, for example, at least one compound chosen from the group consisting of preservatives, emollients, emulsifiers, surfactants, moisturizers, thickeners, conditioners, matifying agents, stabilizers, antioxidants, texturing agents, gloss agents, film-forming agents, solubilizing agents, pigments, dyes, fragrances and sunscreens. These excipients are preferably chosen from the group consisting of amino acids and their derivatives, polyglycerols, esters, cellulose polymers and derivatives, lanolin derivatives, phospholipids, lactoferrins, lactoperoxidases, sucrose-based stabilizers, vitamin E and its derivatives, natural and synthetic waxes, vegetable oils, triglycerides, unsaponifiable compounds, phytosterols, plant esters, silicones and their derivatives, protein hydrolysates, jojoba oil and its derivatives, lipo/water-soluble esters, betaines, aminoxides, plant extracts, sucrose esters, titanium dioxides, glycines, and parabens, and more preferably from the group consisting of steareth-2, steareth-21, glycol-15 stearyl ether, cetearyl alcohol, phenoxyethanol, methylparaben, ethylparaben, propylparaben, butylparaben, butylene glycol, caprylyl glycol, natural tocopherols, glycerin, sodium dihydroxycetyl phosphate, isopropyl hydroxycetyl ether, glycol stearate, triisononanoin, octyl cocoate, polyacrylamide, isoparaffin, laureth-7, a carbomer, propylene glycol, hexylene glycol, glycerol, bisabolol, a dimethicone, sodium hydroxide, PEG-30 dipolyhydroxystearate, capric/caprylic triglycerides, cetearyl octanoate, dibutyl adipate, grape seed oil, jojoba oil, magnesium sulfate, EDTA, a cyclomethicone, xanthan gum, citric acid, sodium lauryl sulfate, mineral waxes and oils, isostearyl isostearate, propylene glycol dipelargonate, propylene glycol isostearate, PEG-8, beeswax, hydrogenated palm kernel oil glycerides, lanolin oil, sesame oil, cetyl lactate, lanolin alcohol, castor oil, titanium dioxide, lactose, sucrose, low-density polyethylene, and an isotonic saline solution.

The cosmetic composition according to the invention can be chosen from an aqueous or oily solution, an aqueous cream or gel or an oily gel, in particular a shower gel; a shampoo; a milk; an emulsion, a microemulsion or a nanoemulsion, which is in particular oil-in-water or water-in-oil or multiple or silicone-based; a mask; a serum; a lotion; a liquid soap; a dermatological bar; an ointment; a foam; a patch; an anhydrous product, which is preferably liquid, pasty or solid, for example in the form of makeup powders, of a wand or of a stick, in particular in the form of a lipstick. Advantageously, it is a cream or a serum, in particular an eye or lip contour.

The compositions according to the invention are more particularly applied to the face, preferably daily, preferably once or twice a day, preferably in the morning and/or the evening.

Advantageously, the cosmetic compositions according to the invention contain other ingredients of interest, in particular cosmetic interest, preferably agents which have similar properties. Preferably, these are the conventional ingredients of anti-aging compositions and/or compositions which improve the density and/or the firmness of the skin and/or mucous membranes and/or which improve the complexion of the skin and/or cutaneous homeostasis, in particular those chosen from filling agents, tightening agents, moisturizing agents, and agents for stimulating extracellular matrix molecules.

The cosmetic compositions according to the invention can also contain cosmetic active ingredients which result in a supplementary or optionally synergistic effect, such as moisturizing active agents, anti-aging active agents, free-radical scavenging active agents, fibroblast growth factor (FGF) protecting agents, agents which stimulate fibroblast activity and/or proliferation and/or thermal waters. They can thus be, for example, skin-coloring or propigmenting agents, NO-synthase inhibitors, antiseborrheic agents for oily-skin care, agents which stimulate the synthesis of dermal or epidermal macromolecules, in particular of the extracellular matrix, and/or which prevent their degradation, for a synergistic or supplementary effect, agents which stimulate fibroblast or keratinocyte proliferation and/or keratinocyte differentiation, for a synergistic or supplementary effect, antimicrobial agents, tightening agents, antipollution agents or free-radical scavengers, soothing, calming or relaxing agents, agents which act on the microcirculation in order to improve the radiance of the complexion, in particular of the face, for a synergistic or supplementary effect, photoprotective agents, healing agents, slimming agents, anti-aging agents, for a synergistic or supplementary effect, or optionally moisturizing agents and/or agents which reinforce the epidermal barrier.

The moisturizing, emollient or humectant active agents can reinforce the barrier function and reduce imperceptible water losses and/or increase the water content of the skin and/or of the mucous membranes or stimulate the secretory activity of the sebaceous glands and/or stimulate the synthesis of aquaporin in order to improve the circulation of water in the cells. By way of nonlimiting example, mention may be made of the following active agents: serine, urea and its derivatives, the products sold under the name Marine Filling Spheres™, Advanced Moisturizing Complex™, Hyaluronic Filling Spheres™, Vegetal Filling Spheres™, Osmogelline™ or Micropatch™, alkylcelluloses, lecithins, sphingoid-based compounds, ceramides, phospholipids, cholesterol and its derivatives, glycosphingolipids, phytosterols (stigmasterol and β-sitosterol, campesterol), essential fatty acids, 1,2-diacylglycerol, 4-chromanone, pentacyclic triterpenes, such as ursolic acid, petrolatum, lanolin, sugars, in particular trehalose and its derivatives, rhamnose, fructose, maltose, lactose, erythritol, mannitol, D-xylose and glucose, adenosine and its derivatives, sorbitol, polyhydric alcohols, advantageously C₂-C₆ and more advantageously C₃-C₆ polyhydric alcohols, such as glycerin, propylene glycol, dipropylene glycol, diglycerin, polyglycerin and their mixtures, glycerol and its derivatives, glyceryl polyacrylate, sodium lactate, pentanediol, serine, lactic acids, AHAs, BHAs, sodium pidolate, xylitol, sodium lactate, ectoin and its derivatives, chitosan and its derivatives, collagen, plankton, steroidal derivatives (including DHEA, its 7-oxidized and/or 17-alkylated derivatives and sapogenins), methyl dihydrojasmonate, vitamin D and its derivatives, a Malva sylvestris extract or a Centella asiatica extract, acrylic acid homopolymers, beta-glucan and in particular sodium carboxymethyl beta-glucan, a C-glycoside derivative, such as those described in the application WO 02/051828, a musk rose oil, an extract of the microalga Porphyridium cruentum enriched in zinc, sold by Vincience under the name Algualane Zinc™, arginine, the acetyl hexapeptide sold by Lipotech under the name Diffuporine™, the Viola tricolor hydrolysate sold by Silab under the name Aquaphyline™.

The active agent can also be chosen from anti-aging agents, that is to say agents having in particular a restructuring effect on the skin barrier, agents which prevent and/or reduce the glycation of skin proteins, in particular dermal proteins, such as collagen, active agents which stimulate the energy metabolism of cells, and their mixtures, an agent with an overall anti-aging action, in particular niacinamide or vitamin B3 and derivatives. The agent having a restructuring effect on the skin barrier can be chosen from one of the yeast extracts, such as Relipidium™ from BASF Beauty Care Solutions France SAS, sphingosines, such as salicyloyl sphingosine, a mixture of xylitol, xylityl polyglycoside and xylitan, extracts of Solanaceae, such as Lipidessence™ from BASF Beauty Care Solutions France SAS, and their mixtures. Mention may also in particular be made of ceramides, sphingoid-based compounds, glycosphingolipids, phospholipids, cholesterol and its derivatives, phytosterols, essential fatty acids, diacylglycerol, 4-chromanone and chromone derivatives and their mixtures, vitamin B5 or pantothenate and derivatives.

The active agent which stimulates the energy metabolism of cells can, for example, be chosen from biotin, a mixture of sodium, manganese, zinc and magnesium salts of pyrrolidonecarboxylic acid, a mixture of zinc gluconate, copper gluconate and magnesium gluconate, and their mixtures.

The antiseborrheic agent in the composition according to the invention can be a 5α-reductase inhibitor, such as retinoids, sarcosine, zinc salts, in particular zinc gluconate or zinc salicylate, azelaic acid and/or their derivatives, and/or their mixtures, and an Orthosiphon stamineus extract sold under the name MAT XS™ Bright by BASF Beauty Care Solutions France SAS.

The composition can also contain a sebum-absorbing agent, in particular a talc and/or an absorbent polymer, an antibacterial agent, in particular those described in the patent application FR 2 863 893, and in particular a Boldo extract, such an extract being in particular sold by the applicant company under the name Betapur™, a comedolytic agent, in particular retinoic acid and one of its derivatives, such as isotretinoin, adapalene and/or 13-cis-retinoic acid and benzoyl peroxide, a local antibiotic agent, in particular erythromycin and/or clindamycin phosphate and their mixtures.

Among the active agents which stimulate the synthesis of dermal macromolecules or which prevent their degradation, mention may be made of those which act as:
an agent which stimulates fibronectin synthesis, in particular a corn extract, such an extract being in particular sold by the applicant company under the name Deliner™, and the palmitoyl pentapeptide sold by Sederma under the trade name Matrixil™,
an agent for protecting extracellular matrix fibroblast growth factor (FGF2) against its degradation and/or its denaturation, in particular a Hibiscus abelmoschus extract as described in the patent application in the name of the applicant company filed under number FR 0 654 316, and/or an agent for stimulating fibroblast growth, for example a fermented soya extract containing peptides, known under the name Phytokine™, sold by the applicant company and also described in the patent application EP 1 119 344 B1 (Laboratoires Expanscience), and preferably a combination of these two extracts;
an agent which stimulates laminin synthesis, in particular an extract of malt modified by biotechnology, such an extract being in particular sold by the applicant company under the name Basaline™;
an agent which stimulates the expression and/or activity of hyaluronan synthase 2 (HAS2), such as the plant extracts described in the patent application FR 2 893 252 A1 and in particular an aqueous extract of galanga (Alpinia galanga);
an agent which stimulates the synthesis of lysyl oxidase like (LOXL), such as a Geophila cordifolia extract and those described in the patent application FR 2 855 968, in particular a dill extract;
an agent which stimulates intracellular ATP synthesis, in particular an extract of the alga Laminaria digitata;
an active agent which stimulates glycosaminoglycan synthesis, such as the product of milk fermentation;
a collagen-stimulating active agent, such as retinol and/or vitamin C;
an active agent which inhibits metalloproteinases (MMPs), such as more particularly MMP 1, 2, 3 and 9, such as retinoids and derivatives, oligopeptides and lipopeptides, lipoamino acids, the malt extract sold by BASF Beauty Care Solutions France under the trade name Collalift™, the hydrolyzed potato extract sold under the name Extracellium™ by BASF Beauty Care Solutions France SAS; lycopene; isoflavones, quercetin, kaempferol and apigenin.

The agents which stimulate keratinocyte proliferation which can be used in the composition according to the invention comprise in particular retinoids, such as retinol and its esters, including retinyl palmitate, and phloroglucinol. The agents which stimulate keratinocyte differentiation comprise, for example, minerals, such as calcium, and lignans, such as secoisolariciresinol, and also the Achillea millefolium extract sold under the name Neurobiox™ by BASF Beauty Care Solutions France.

The antimicrobial agents capable of being used in the composition according to the invention can in particular be chosen from 2,4,4′-trichloro-2′-hydroxydiphenyl ether (or triclosan), 3,4,4′-trichlorocarbanilide, phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, octoxyglycerin, octanoylglycine, caprylyl glycol, 10-hydroxy-2-decanoic acid, farnesol, phytosphingosines, and their mixtures.

Among the tightening agents which can be used in the composition according to the present invention, mention may in particular be made of synthetic polymers, such as polyurethane latexes or acrylic latexes; polymers of natural origin, in particular polyholosides in the form of starch or in the form of carrageenans, alginates, agars, gellans, cellulose-based polymers and pectins; soya vegetable proteins and protein hydrolysates; mixed silicates; wax microparticles; colloidal particles of inorganic filler which are chosen, for example, from silica and silica-alumina composites; and also their mixtures.

The composition can comprise “antipollution”' agents, in particular ozone scavengers, which are, for example, vitamin C and its derivatives, including ascorbyl glucoside; phenols and polyphenols, in particular tannins, ellagic acid and tannic acid; epigallocatechin and the natural extracts containing it, in particular green tea extracts; anthocyans; phenol acids, stilbenes; active agents which are scavengers of mono- or polycyclic aromatic compounds, tannins, such as ellagic acid, and indole derivatives, and/or active agents which scavenge heavy metals, such as EDTA, free-radical scavenging active agents, such as vitamin E and its derivatives, such as tocopheryl acetate; bioflavonoids; coenzyme Q10 or ubiquinone.

As soothing agents which can be used in the composition according to the invention, mention may be made of: pentacyclic triterpenes, ursolic acid and its salts, oleanolic acid and its salts, betulinic acid and its salts, salicylic acid salts and in particular zinc salicylate, bisabolol, allantoin, unsaturated omega-3 oils, cortisone, hydrocortisone, indomethacin and betamethasone, anti-inflammatory active agents and in particular those described in the application FR 2 847 267, especially the Pueraria lobata root extract sold under the name Inhipase™ by BASF Beauty Care Solutions France SAS, Theobroma cacao extracts.

The active ingredients which act on the microcirculation (vasoprotectors or vasodilators) can be chosen from flavonoids, ruscogenins, nicotinates and essential oils.

The photoprotective active agent or UV-A- and/or UV-B-screening agent ingredients which can be used according to the present invention are in particular protoprotective agents which are active in the UV-A and/or UV-B region(s), such as para-aminobenzoic acid derivatives, in particular Uvinul P25™, sold by BASF, salicylic derivatives, in particular homosalate, alone or in association with titanium oxides, dibenzoylmethane derivatives, cinnamic derivatives, diphenylacrylate derivatives, including octocrylene, sold in particular under the trade name Uvinul N539™ by BASF, benzophenone derivatives, in particular benzophenone-1, sold in particular under the trade name Uvinul 400™ by BASF, benzylidenecamphor derivatives, benzimidazole derivatives, triazine derivatives, including ethylhexyl triazone, sold in particular under the trade name Uvinul T150™ by BASF, benzotriazole derivatives, anthranilic derivatives, imidazoline derivatives, benzalmalonate derivatives, 4,4-diarylbutadiene derivatives, and their mixtures.

The active agents which provide an effect of well-being, such as those which mimic the effects of β-endorphins for improving the barrier function of the skin, such as those mentioned in the patent application US 2006069032; active agents which stimulate the synthesis of β-endorphins, such as an extract of the plant Tephrosia purpurea.

The slimming active agents can in particular be chosen from: agents which inhibit lipoprotein lipase, such as those described in the patent US 2003086949 (Coletica) and in particular an extract of Peruvian liana (Uncaria tomentosa); draining active agents, in particular hesperetin laurate (Flavagrum™) or quercetin caprylate (Flavenger™); agents which inhibit the enzyme phosphodiesterase, agents which activate adenylate cyclase, cAMP and/or active agents capable of trapping spermine and/or spermidine. By way of example of these active agents, mention may be made of a Coleus forskohlii root extract, a Cecropia obtusa extract, a Uva lactuca extract, caffeine, forskolin, theophylline, theobromine and/or their derivatives, a hydrolyzed kappa carrageenan product called Slimexcess™ sold by BASF Beauty Care Solutions France SAS, and/or their mixtures.

In a specific embodiment, the cosmetic composition according to the present invention does not contain any depigmenting and/or anti-tyrosinase and/or melanogenesis-inhibiting agent.

Numerous topically acceptable and active cosmetic ingredients are known by a person skilled in the art for improving the health and/or the physical appearance of the skin and/or mucous membranes and/or scalp. A person skilled in the art knows how to formulate cosmetic compositions in order to obtain the best effects.

Furthermore, the compounds described in the present invention can have a synergistic effect when they are combined with one another. These combinations are also covered by the present invention. The CTFA Cosmetic Ingredient Handbook, Second Edition (1992), describes various cosmetic and pharmaceutical ingredients commonly used in the cosmetics industry, which are in particular suitable for topical use. Examples of these classes of ingredients include, without being limited thereto, the following compounds: abrasive; absorbents; compound for esthetic purposes, such as fragrances; pigments; dyes; essential oils; astringents, such as clove oil, menthol, camphor, eucalyptus oil, eugenol, menthyl lactate or witch hazel distillate; anti-acne agents; antiflocculants; antifoaming agents; antimicrobial agents, such as iodopropyl butylcarbamate; antioxidants, such as ascorbic acid; binders; biological additives; buffers; swelling agents; chelating agents; additives; biocidal agents; denaturing agents; thickeners; vitamins; film-forming materials; polymers; opacifying agents; pH adjusters; reducing agents; conditioning agents, such as humectants, and derivatives or equivalents of these.

In yet another specific embodiment of the present invention, the Tapirira guianensis extract according to the invention, preferably obtained by aqueous extraction, is dissolved in a solvent, in particular a polar solvent, such as water, an alcohol, a polyol, a glycol or one of their mixtures, preferably an aqueous/glycolic mixture, more preferably containing a glycol chosen from caprylyl glycol, hexylene glycol and their mixtures.

Particularly advantageously, the extract according to the invention is solubilized in an aqueous solution comprising hexylene glycol, caprylyl glycol or their mixture, advantageously hexylene glycol and caprylyl glycol. Advantageously, the aqueous solution in which the Tapirira guianensis extract according to the invention is solubilized comprises hexylene glycol, in particular between 0.1 and 10% by weight of hexylene glycol, with respect to the total weight of the aqueous solution, more particularly between 1 and 5% by weight of hexylene glycol, with respect to the total weight of the aqueous solution.

In particular, the aqueous solution in which the Tapirira guianensis extract according to the invention is solubilized comprises caprylyl glycol, in particular between 0.01 and 5% by weight of caprylyl glycol, with respect to the total weight of the aqueous solution, more particularly between 0.1 and 1% by weight of caprylyl glycol, with respect to the total weight of the aqueous solution.

Particularly advantageously, the aqueous solution in which the Tapirira guianensis extract according to the invention is solubilized comprises hexylene glycol and caprylyl glycol, in particular in the proportions indicated above.

Advantageously, the Tapirira guianensis extract according to the invention, preferably obtained by aqueous extraction, is solubilized in the aqueous solution in a content of between 0.1 and 10% by weight, with respect to the total weight of the aqueous solution, in particular of between 0.5 and 5% by weight, with respect to the total weight of the aqueous solution. In particular, this aqueous solution comprises hexylene glycol and caprylyl glycol, advantageously in the proportions indicated above for these components.

The aqueous solution in which the extract according to the invention is solubilized can comprise a thickening and/or structuring agent, such as xanthan gum, advantageously in a content of between 0.01 and 5% by weight, with respect to the total weight of the aqueous solution, in particular between 0.1 and 1% by weight, with respect to the total weight of the aqueous solution.

The present invention also relates to a cosmetic care method, characterized in that it comprises the application, to at least one concerned area of healthy skin and/or healthy mucous membrane and/or healthy scalp, in particular of a human being, of a Tapirira guianensis extract or of a cosmetic composition comprising such an extract for stimulating the expression of perlecan and/or dystroglycan and/or collagen XVIII and/or VE-cadherin and/or claudin-5, in particular in the extracellular matrix and/or in the epithelial basement membrane, especially the dermoepidermal junction.

Advantageously, this cosmetic care method is for preventing and/or combating aging of the skin and/or mucous membranes and/or scalp, in particular chronobiological and/or photobiological aging, for preventing and/or combating the decrease in homeostasis of the skin and/or mucous membranes and/or scalp and/or for improving it, in particular in the epidermis, for reinforcing the epithelial basement membrane of the skin and/or mucous membranes and/or scalp, preferably the dermoepidermal junction, for improving keratinocyte proliferation and/or differentiation, especially at the epidermal level, in particular associated with aging of the skin and/or mucous membranes and/or scalp, for preventing and/or combating a decrease in vascularization of the skin and/or mucous membranes and/or scalp and/or for improving it, in particular for improving the structure of the capillaries of the skin and/or mucous membranes and/or scalp, in particular skin capillaries, for improving the morphogenesis of the epithelium of the skin and/or mucous membranes and/or scalp, preferably the epidermis, for restoring the epithelial architecture of the skin and/or mucous membranes and/or scalp, preferably the epidermal architecture, in particular of the skin and/or mucous membranes and/or scalp having undergone aging, in particular chronobiological aging, for improving the complexion of the skin and/or mucous membranes, especially making it uniform, for improving the firmness and/or the density of the skin and/or mucous membranes and/or scalp, and/or for combating the decrease in the thickness of the epithelium of the skin and/or mucous membranes and/or scalp, preferably the epidermis, and/or for increasing the thickness of the epithelium of the skin and/or mucous membranes and/or scalp, preferably the epidermis, and/or the treatment and/or the prevention of cracks, and/or for combating and/or treating and/or preventing water retention and/or cellulite and/or periorbital bags and/or for increasing and/or maintaining cell adhesion.

The present invention also relates to a cosmetic composition, advantageously intended for application by the topical route, characterized in that it comprises a Tapirira guianensis extract, in particular as defined above, and an advantageously topically acceptable cosmetic excipient. Advantageously, the cosmetic composition is as defined above.

Another subject matter of the present invention is a cell culture medium, in particular a medium for the culturing of cultured endothelial cells and/or keratinocytes, and/or three-dimensional models containing them, such as reconstructed epidermis, capillary, vessel or skin models, comprising the Tapirira guianensis extract according to the invention, in particular as defined above, and advantageously a compound chosen from fetal calf serum, the pituitary extract, or a hormone, an amino acid, a sugar, a growth factor, a recombinant protein and their mixtures.

In a first specific embodiment of the invention, the extract according to the invention is added directly to the culture medium according to the invention during the manufacture of said medium.

In a second specific embodiment of the invention, the extract according to the invention is added extemporaneously to the culture medium according to the invention.

In a third specific embodiment of the invention, the extract according to the invention is added extemporaneously in combination with a compound chosen from the group consisting of fetal calf serum, a pituitary extract, a hormone, an amino acid, a sugar, a growth factor, a recombinant protein and their mixtures.

The culture medium according to the invention can be intended for the culturing of keratinocytes or endothelial cells for medical, pharmaceutical or dermatological applications.

The addition of the Tapirira guianensis extract is likely to facilitate and/or shorten the culturing stages and/or to improve the quality of the cultures and cell construction (cell layer, pseudoepidermis, reconstructed epithelia).

Preferably, the culture medium according to the invention comprises a Tapirira guianensis extract at a concentration of between 1×10⁻⁴ and 10% by weight, with respect to the total weight of the culture medium, preferably between 0.001 and 0.1% by weight, with respect to the total weight of the culture medium.

A subject matter of the present invention is thus the use of the Tapirira guianensis extract according to the invention in a culture medium and/or of the culture medium comprising the extract for stimulating the expression of perlecan and/or dystroglycan and/or VE-cadherin and/or claudin-5 and/or collagen XVIII, in particular in the extracellular matrix and/or in the epithelial basement membrane, especially the dermoepidermal junction, preferably in cultured cells, in particular chosen from endothelial cells and keratinocytes.

Other aims, characteristics and advantages of the invention will become clearly apparent to a person skilled in the art following the reading of the figures and of the explanatory description, which refers to examples which are given only by way of illustration and which cannot in any way limit the scope of the invention.

The examples form an integral part of the present invention and any characteristic which appears novel with respect to any prior state of the art following the description taken in its entirety, including the examples, forms an integral part of the invention in its function and its general nature.

Thus, each example has a general scope.

Moreover, in the examples and unless otherwise indicated, the temperature is expressed in degrees Celsius and the pressure is atmospheric pressure.

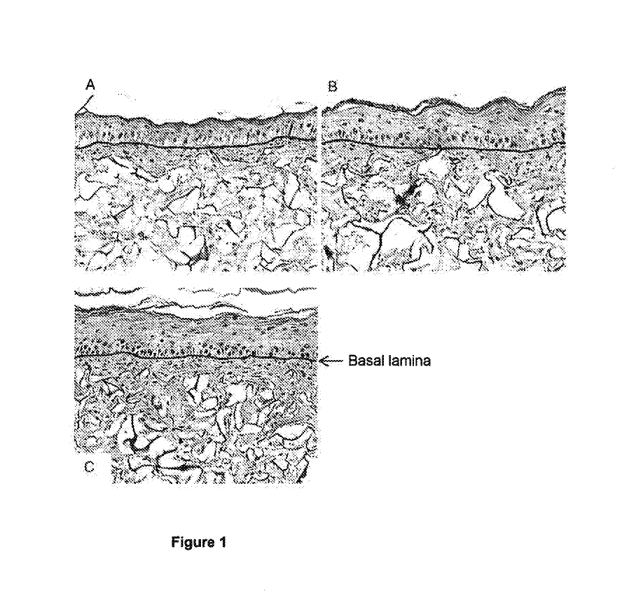

FIG. 1 represents the effect of a Tapirira guianensis extract according to the invention on the thickness of the epidermis (example 5). FIG. 1A represents a section observed in microscopy of the untreated reconstructed skin model, FIG. 1B represents that of one and the same model treated with a 0.05% (w/w) Tapirira guianensis extract and FIG. 1C represents that of a model treated with a 0.1% (w/w) Tapirira guianensis extract.

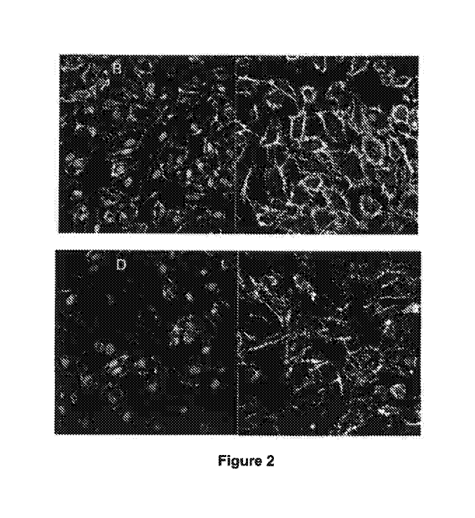

FIG. 2 represents the effect of a Tapirira guianensis extract according to the invention on the protein expression of VE-cadherin (FIGS. 2A and B) and claudin-5 (FIGS. 2C and D) at the cell junctions of human microvascular endothelial cells after immunolabeling (A and C. Untreated cells; B and D. Cells treated with a 0.8% Tapirira guianensis extract according to the invention) (Example 6).

### EXAMPLE 1

### Preparation of Tapirira guianensis Extract According to the Invention by Aqueous Extraction

a) Tapirira guianensis leaves were ground and then macerated in water at ambient temperature, that is to say between 18 and 25° C., in this instance at approximately 20° C., for two hours, the content of ground Tapirira guianensis leaves being 1% by weight, with respect to the plant/water total weight.

The insoluble fractions were separated by filtration at 0.45 μm and the liquid which contains the aqueous extract according to the invention is recovered.

b) Tapirira guianensis leaves were ground and then macerated in water at 1% (w/w), at a temperature preferably of between 0 and 20° C., preferably at 4° C.

The duration of maceration is advantageously between 30 min and 24 hours, with stirring, in this instance 16 hours.

The solution is centrifuged, preferably at 8000 rpm for 10 min and the supernatant is recovered. The supernatant is ultrafiltered on filters having different cutoff thresholds and in particular a 0.22 μm filter.

The extract thus obtained can be used directly in liquid form. This extract was tested at different dosages in the final culture medium in the following examples 2 to 6.

This extract can also be formulated in the form of a cosmetic ingredient, as presented in example 7.

c) Tapirira guianensis leaves were ground and then macerated at 1% (w/w) in a 75%/25% water/butylene glycol mixture at a temperature preferably of between 0 and 20° C., in this instance at 4° C. The duration of maceration is advantageously between 30 min and 24 hours, with stirring, in this instance 10 hours.

The solution is centrifuged, preferably at 8000 rpm for 10 min, and the supernatant is recovered. The supernatant is ultrafiltered on filters having different cutoff thresholds and in particular a 0.45 μm filter.

The extract thus obtained is subsequently dried, in particular on a support of maltodextrin type, and then resolubilized in water at 1% (w/w).

### EXAMPLE 2

### Effect of a Tapirira guianensis Extract According to the Invention on the Protein Expression of Perlecan in Keratinocytes

Protocol

A fluoroimmunoassay (FIA) test was carried out and consists in revealing the antigen of interest, in this instance perlecan, by fluorescence. This method is semi-quantitative, highly sensitive and reproducible, and exhibits the advantage of detecting the protein of interest in its native form in its environment, without a denaturation process. The keratinocytes resulting from an abdominal skin biopsy on donors aged 30, 50 and 61 years are extracted and attached to the bottom of the wells at a density of 5000 cells per well, and grow for 3 days in a complete medium, that is to say a medium containing fetal calf serum (FCS), and then for 16 hours in a defined medium without FCS. They are subsequently cultured for 48 hours, either with the extract obtained in example 1b), tested at different dosages as percentage by weight in the final culture medium, or without the extract, termed control. The cells are then washed in phosphate buffered saline (PBS), before being fixed, permeabilized and unmasked. Before applying the (anti-perlecan) primary antibody for 90 minutes, the cells are saturated with BSA (bovine serum albumin) at 1% for 1 hour. After washes in PBS buffer, the secondary antibody linked to the fluorocrome FITC (fluorescein isothiocyanate) is incubated for 2 hours. The cells are then washed in PBS buffer, and then solubilized with 20 mM of ammonium hydroxide and 0.5% of 100% Triton. The fluorescence is then read on a spectrofluorimeter with the appropriate filters. The results are collated in table 1 below. The experiment is carried out six times (n=6). The values in the table represent the values as percentage with respect to the nontreated control cells. The values represent the mean of several experiments on different batches of extracts. “Mean” denotes the mean and “SD” denotes the standard deviation.

TABLE 1
Percentage of protein expression of perlecan in
keratinocytes from donors aged 30, 50 and 61 years
as a function of the dose of extract used

| | | Mean | SD |
|---|---|---|---|
| | Control | 100 | 11.5 |
| 30 | 0.20% Tapirira guianensis extract | 171.4 | 15.0 |
| years | 0.40% Tapirira guianensis extract | 177.5 | 18.9 |
| | 0.80% Tapirira guianensis extract | 195.9 | 15 |
| | 1.0% Tapirira guianensis extract | 183.7 | 20.1 |
| 50 | 0.20% Tapirira guianensis extract | 144.2 | 14.1 |
| years | 0.40% Tapirira guianensis extract | 184.6 | 17.9 |
| | 0.80% Tapirira guianensis extract | 196.1 | 17.9 |
| | 1.0% Tapirira guianensis extract | 190.4 | 18.9 |
| 61 | 0.20% Tapirira guianensis extract | 150.0 | 10.6 |
| years | 0.40% Tapirira guianensis extract | 163.0 | 13.1 |
| | 0.80% Tapirira guianensis extract | 169.6 | 11.9 |
| | 1.0% Tapirira guianensis extract | 189.1 | 8.4 |

Conclusions:

The extract according to the invention induced a significant increase in the protein expression of perlecan in keratinocytes. This increase in the protein expression was observed whatever the age of the donor.

The extract according to the invention thus induces an improvement in the structural cohesion of the epithelium, preferably the epidermis.

### EXAMPLE 3

### Effect of a Tapirira guianensis Extract According to the Invention on the Protein Expression of Perlecan in Endothelial Cells

The technique is the same as in example 2, except that it is carried out on endothelial cells extracted from abdominal skin biopsy on donors aged 38 years or 28 years.

The results are collated in tables 2 and 3 below; “Mean” denotes the mean and “SD” denotes the standard deviation.

TABLE 2
Percentage of expression of perlecan in the endothelial cells
of a 38-year-old donor as a function of the dose of extract used
n = 6; the Tapirira guianensis extract is obtained
according to example 1b) and is tested at different dosages
as percentage by weight in the final culture medium.

| | Mean | SD |
|---|---|---|
| Control | 100 | 18.2 |
| 0.1% Tapirira guianensis extract | 145.7 | 6.5 |
| 0.2% Tapirira guianensis extract | 147.2 | 11.3 |
| 0.4% Tapirira guianensis extract | 160.4 | 11.1 |
| 0.8% Tapirira guianensis extract | 238.6 | 31.4 |

TABLE 3
Percentage of protein expression of perlecan in the endothelial
cells of a 28-year-old donor as a function of the active agent
used n = 6; the Tapirira guianensis extract is obtained
according to example 1b) and is tested at different dosages
as percentage by weight in the final culture medium.

| | Mean | SD |
|---|---|---|
| Control | 100 | 10 |
| 0.1% Tapirira guianensis aqueous extract | 152.2 | 5.8 |
| 0.2% Tapirira guianensis aqueous extract | 150.0 | 15.5 |
| 0.4% Tapirira guianensis extract | 182.0 | 18.1 |
| 0.8% Tapirira guianensis extract | 164.3 | 12.7 |

Conclusions:

The extract according to the invention significantly increased, at the doses tested, the protein expression of perlecan in the endothelial cells, which testifies to its properties in improving the microvascular structural cohesion. This increase was observed whatever the age of the donors.

### EXAMPLE 4

### Effect of a Tapirira guianensis Extract According to the Invention on the Protein Expression of Dystroglycan in Keratinocytes

The technique is the same as in example 2 except that the antigen of interest is in this instance dystroglycan and the antibody used is an anti-dystroglycan.

The results are collated in table 4 below; “Mean” denotes the mean and “SD” denotes the standard deviation.

TABLE 4
Percentage of protein expression of dystroglycan in the keratinocytes
of a 50-year-old donor as a function of the dose of extract used
n = 6; the Tapirira guianensis extract is obtained according
to example 1b) and is tested at different dosages as percentage
by weight in the final culture medium.

| | Mean | SD |
|---|---|---|
| Control | 100 | 9.7 |
| 0.2% Tapirira guianensis extract | 119.0 | 7.6 |
| 0.4% Tapirira guianensis extract | 121.3 | 5.9 |

Conclusions:

The extract according to the invention significantly increased, at the doses tested, the protein expression of dystroglycan in the keratinocytes. The extract according to the invention thus induces an improvement in the structural cohesion of the epithelium, preferably the epidermis.

### EXAMPLE 5

### Effect of th Tapirira guianensis Extract According to the Invention on the Thickness of the Epidermis

A Tapirira guianensis aqueous extract prepared according to example 1b) was tested at final concentrations of 0.05% and 0.1% by weight, with respect to the total weight of the culture medium according to the invention, for its effect on the morphogenesis of the epidermis.

The extract according to the invention was tested on a reconstructed skin model of Mimeskin® type known to a person skilled in the art.

In brief, the skin model is obtained by culturing fibroblasts for 35 days, from which stage epidermal differentiation takes place and the fibroblasts differentiate to give keratinocytes.

After culturing for 35 days, the cells were rinsed with a phosphate buffered saline (PBS) buffer containing calcium, magnesium and antibiotics and then fixed in cold methanol for 10 minutes.

Sections were taken and the thickness of the epidermis was measured in μm in the samples treated with the extract according to the invention or without (Control) (table 5).

TABLE 5
Thickness of the epidermis (μm) measured in the samples treated
or not treated with the extract according to the invention (n =
4); “Mean” denotes the mean and “SD” denotes the standard deviation

| | Mean | SD |
|---|---|---|
| Control | 49.12 | 2.8 |
| 0.05% Tapirira guianensis extract | 64.9 | 4.7 |
| 0.1% Tapirira guianensis extract | 73.8 | 2.8 |

Conclusions:

An increase in the thickness of the epidermis was observed when the cells differentiated to give keratinocytes were treated with an extract according to the invention (FIG. 1). This effect was observed at each of the concentrations tested (as percentage by weight with respect to the total weight of the culture medium according to the invention).

### EXAMPLE 6

### Effect of a Tapirira guianensis Extract According to the Invention on the Protein Expression of VE-Cadherin and Claudin-5 at the Cell Junctions of Human Microvascular Endothelial Cells

Materials and Methods:

Microvascular endothelial cells resulting from a blood biopsy on an adult donor aged 50 years were inoculated in a complete culture medium in the presence of 5% of FBS (fetal bovine serum) for 10 days.

When the cells reached subconfluence, the confluence corresponding to the cell stage for which 100% of the cultured cells adhere to one another, the cells were inoculated in a culture chamber containing an EGM2-MV culture medium (endothelial cell culture medium) containing 1% of FBS (by weight, with respect to the total weight of the culture medium) for 48 hours, in or not in the presence of a Tapirira guianensis extract according to the invention (prepared according to example 1b)), at a final concentration of 0.8% by weight with respect to the weight of the culture medium.

The cells were then washed in a phosphate buffered saline buffer and then fixed with glacial methanol for 10 min. An anti-VE-cadherin antibody was added to the culture medium for the purpose of carrying out an immunolabeling. The slides were subsequently incubated with a second antibody coupled directly to an anti-claudin-5 antibody.

After three washes in PBS, the cells were observed in confocal microscopy.

Conclusions:

The confocal microscopy results are presented in FIG. 2.

The results show that the Tapirira guianensis extract according to the invention increases the protein expression of VE-cadherin and claudin-5 at the intercellular junctions. The Tapirira guianensis extract thus promotes cell adhesion at the dermoepidermal junction.

### EXAMPLE 7

### Composition Comprising the Extract According to Present Invention Intended to be Incorporated in a Cosmetic Composition (Cosmetic Ingredient)

The Tapirira guianensis extract is obtained according to example 1b) and is mixed with the other ingredients of the following formulation:

| | % by weight with respect to the |
|---|---|
| Ingredient | total weight of the composition |
| Water | >50% |
| Tapirira guianensis aqueous | 0.5-5% |
| extract (Ex. 1b)) | |
| Hexylene glycol | 1-5% |
| Caprylyl glycol | 0.1-1% |
| Xanthan gum | 0.1-1% |

### EXAMPLE 8

### Compositions Containing the Tapirira guianensis Extract According to the Invention

Methods known to a person skilled in the art are used to mix together the different parts A, B, C, D, E or F in order to prepare a composition according to the present invention.

The “products of the invention” represent a Tapirira guianensis extract preferably obtained according to example 1b).

The products of the invention can also be provided in the form of liposomes containing 5% of soybean lecithin and incorporating a solution of quaternized soybean (600 g in the end) obtained according to the following method of preparation:

30 g of soybean lecithin, 12 g of quaternized soybean solution and 1.5 g of Tapirira guianensis extract prepared according to example 1b) are introduced into a sample tube and diluted in 447 g of pure laboratory water.

After magnetic stirring for 10 minutes at ambient temperature, the mixture is vigorously homogenized for 10 minutes, thus producing a liposomal solution in which the liposomes have a mean size which can vary between 100 and 800 nanometers, according to the exact conditions of homogenization.

The suspension is subsequently left under gentle stirring for one hour. 90 g of butylene glycol, 6 g of phenoxyethanol and 6 g of hydroxyethylcellulose (gelling agent) are subsequently added.

Cosmetic formulation 8a:

| A | Water | q.s. for 100 |
|---|---|---|
| | Butylene Glycol | 2 |
| | Glycerin | 3 |
| | Sodium Dihydroxycetyl Phosphate, Isopropyl | 2 |
| | Hydroxycetyl Ether | |
| B | Glycol Stearate SE | 14 |
| | Triisononanoin | 5 |
| | Octyl Cocoate | 6 |
| C | Butylene Glycol, Methylparaben, Ethylparaben, | 2 |
| | Propylparaben, pH adjusted to 5.5 | |
| D | Products of the invention | 0.01-10% |

Cosmetic formulation 8b:

| | | |
|---|---|---|
| A | Water | q.s. for 100 |
| | Butylene Glycol | 2 |
| | Glycerin | 3 |
| | Polyacrylamide, Isoparaffin, Laureth-7 | 2.8 |
| B | Butylene Glycol, Methylparaben, Ethylparaben, | 2 |
| | Propylparaben | |
| | Phenoxyethanol, Methylparaben, Propylparaben, | 2 |
| | Butylparaben, Ethylparaben | |
| | Butylene Glycol | 0.5 |
| D | Products of the invention | 0.01-10% |

Cosmetic formulation 8c:

| | | |
|---|---|---|
| A | Carbomer | 0.50 |
| | Propylene Glycol | 3 |
| | Glycerol | 5 |
| | Water | q.s. for 100 |
| B | Octyl Cocoate | 5 |
| | Bisabolol | 0.30 |
| | Dimethicone | 0.30 |
| C | Sodium Hydroxide | 1.60 |
| D | Phenoxyethanol, Methylparaben, Propylparaben, | 0.50 |
| | Butylparaben, Ethylparaben | |
| E | Fragrance | 0.30 |
| F | Products of the invention | 0.01-10% |

Dermatological composition 8d in the form of an ointment:

| | | |
|---|---|---|
| A | Excipients | |
| | Low-density polyethylene | 5.5 |
| | Liquid paraffin | q.s. for 100 |
| B | Product of the invention* | 0.001-0.1 |

*The Tapirira guianensis extract is that described in example 1b), followed by a stage of sterilization and drying.

1.-25. (canceled)

26. A method of cosmetic care for stimulating the expression of perlecan and/or dystroglycan and/or VE-cadherin and/or claudin-5; and/or for preventing and/or combating aging of the skin and/or mucous membranes and/or scalp; and/or for preventing and/or combating the decrease in homeostasis of the skin and/or mucous membranes and/or scalp and/or for improving it; and/or for reinforcing the epithelial basement membrane of the skin and/or mucous membranes and/or scalp; and/or for improving keratinocyte proliferation and/or differentiation; and/or for preventing and/or combating a decrease in vascularization of the skin and/or mucous membranes and/or scalp and/or for improving it; and/or for improving the morphogenesis of the epithelium of the skin and/or mucous membranes and/or scalp; and/or for restoring the epithelial architecture of the skin and/or mucous membranes and/or scalp; and/or for improving the complexion of the skin and/or mucous membranes; and/or for improving the firmness and/or the density of the skin and/or mucous membranes and/or scalp; and/or for combating the decrease in the thickness of the epithelium of the skin and/or mucous membranes and/or scalp; and/or for increasing the thickness of the epithelium of the skin and/or mucous membranes and/or scalp; and/or in the treatment and/or prevention of cracks; and/or for combating and/or treating and/or preventing water retention and/or cellulite and/or periorbital bags and/or for increasing and/or maintaining cell adhesion comprising administering by the topical route to a patient in need thereof of an effective amount of a Tapirira guianensis extract.

27. The method as claimed in claim 26, wherein the Tapirira guianensis extract is obtained by aqueous extraction.

28. The method as claimed in claim 26, wherein the Tapirira guianensis extract is obtained by extraction of the aerial parts of Tapirira guianensis.

29. The method as claimed in claim 26, for stimulating the protein expression of perlecan and/or dystroglycan and/or VE-cadherin and/or claudin-5.

30. The method as claimed in claim 26 for improving the complexion of the skin by eliminating red patches and/or by making the complexion uniform and/or by giving it a luminous, radiant, healthy and/or nourished appearance, a well-looking effect and/or a pinkish radiance.

31. The method as claimed in claim 26 for preventing and/or combating aging of the skin by decreasing or suppressing wrinkles and/or fine lines.

32. The method as claimed in claim 26 for preventing and/or combating the decrease in the homeostasis of the skin and/or mucous membranes and/or scalp and/or for improving the homeostasis of the skin and/or mucous membranes and/or scalp.

33. The method as claimed in claim 26 for increasing and/or maintaining cell adhesion and for combating and/or treating and/or preventing water retention and/or cellulite and/or periorbital bags.

34. The method as claimed in claim 26, wherein the Tapirira guianensis extract is applied by the topical route to at least one concerned area of healthy skin and/or healthy mucous membrane and/or healthy scalp.

35. The method as claimed in claim 34, wherein the concerned area of the skin is chosen from the face, neck, neckline, bust and/or hands.

36. The method as claimed in claim 26, wherein the Tapirira guianensis extract is present in a cosmetic composition comprising a topically acceptable cosmetic excipient.

37. The method as claimed in claim 26, wherein the Tapirira guianensis extract is solubilized in an aqueous solution comprising hexylene glycol, caprylyl glycol or their mixture.

38. The method as claimed in claim 37, wherein the Tapirira guianensis extract is solubilized in a content of between 0.1 and 10% by weight, with respect to the total weight of the aqueous solution.

39. The method as claimed in claim 26 for stimulating the expression of perlecan and/or dystroglycan in keratinocytes and/or endothelial cells of the skin and/or mucous membranes and/or scalp.

40. A cosmetic composition comprising a Tapirira guianensis extract and a topically acceptable cosmetic excipient.

41. The cosmetic composition as claimed in claim 40, in which the extract is present in a content of between 1×10⁻⁴ and 10% by weight, with respect to the total weight of the composition.

42. The cosmetic composition as claimed in claim 40, in which the extract is as defined in claim 27.

43. The cosmetic composition as claimed in claim 40, wherein it is chosen from an aqueous or oily solution, an aqueous cream or gel or an oily gel; a shampoo; a milk; an emulsion, a microemulsion or a nanoemulsion; a mask; a serum; a lotion; a liquid soap; a dermatological bar; an ointment; a foam; a patch; an anhydrous product.

44. A method for the treatment and/or prevention of rosacea, telangiectasia and/or pathologies of the oral and/or ocular mucous membranes comprising the administration by the topical route to a patient in need thereof of an effective amount of a Tapirira guianensis extract or of a dermatological composition comprising a Tapirira guianensis extract and a dermatologically acceptable excipient.

45. The method as claimed in claim 44, wherein the Tapirira guianensis extract is as defined in claim 27.

46. A cell culture medium comprising a Tapirira guianensis extract.

47. The culture medium as claimed in claim 46, wherein the extract is present at a concentration of between 1×10⁻⁴ and 10% by weight, with respect to the total weight of the culture medium.

48. A method for stimulating the expression of perlecan and/or dystroglycan and/or VE-cadherin and/or claudin-5 of cultured cells chosen from endothelial cells and/or keratinocytes comprising the addition of an effective amount of the culture medium as claimed in claim 46 to the cells to be cultured in need thereof.
